(19) United States
(12) Patent Application Publication
(10) Pub. No.: US 2003/0224469 A1
(43) Pub. Date: Dec. 4, 2003

(54) Methods and kits for assays utilizing fluorescence polarization

(76) Inventors: Tonia J. Buchholz, Mountain View, CA (US); Thomas J. Burke, Madison, WI (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/161,482

(22) Filed: Jun. 3, 2002

(51) Int. Cl. G01N 33/53; G01N 33/537; G01N 33/543

(52) U.S. Cl. 435/7.92

(57) ABSTRACT

The invention relates to use of compounds labeled with multiple, spectrally distinct fluorophores in assays that utilize fluorescent polarization to determine if those compounds bind to a binding partner and in assays to determine if those compounds are incorporated into a larger molecule or degraded into smaller components. Fluorescence polarization in such assays is measured at multiple wavelengths corresponding to the wavelengths at which each fluorophore absorbs and emits light. This overcomes any potential interference at a particular wavelength that may be caused by other components in the assay.

### TECHNICAL FIELD OF THE INVENTION

The invention relates to use of compounds labeled with multiple, spectrally distinct fluorophores in assays that utilize fluorescent polarization to determine if those compounds bind to a binding partner and in assays to determine if those compounds are incorporated into a larger molecule or degraded into smaller components. Fluorescence polarization in such assays is measured at multiple wavelengths corresponding to the wavelengths at which each fluorophore absorbs and emits light. This overcomes any potential interference at a particular wavelength that may be caused by other components in the assay.

### BACKGROUND OF THE INVENTION

Fluorescent labels have been used to trace compounds for many years in many different applications. One area where the use of fluorescent labeling of molecules is becoming more predominant is in assay technology and in particular high throughput screening (“HTS”) assay technology. Fluorescent labels are preferred over radioactive labels in general because the former do not require special handling of waste, long detection times or large sample volumes. Moreover, fluorescent labels are adaptable to homogeneous assays that do not require a separation step (centrifugation, filtration, precipitation, electrophoresis, chromatography, etc.) prior to measurement, which are preferred for HTS. Another advantage of fluorescent labels is that each fluorescent molecule is very sensitive to light excitation and can be excited many thousands of times.

There are several ways in which assays may detect fluorescent labels (see A. J. Pope et al., DDT, 4, pp. 350-62 (1999) for a comprehensive review). The most basic detection method is fluorescence intensity, where a sample is excited with light at a wavelength at which the fluorescent label is excited and output is measured at the wavelength at which the label emits. Fluorescence intensity has been utilized in immunoassays, to detect protein-protein binding, receptor binding, nucleic acid hybridization, and localization and visualization of cellular components (through the use of labeled antibodies to those components), etc. Fluorescence intensity has certain drawbacks such as interference by the colorization of samples and the presence of other materials utilized in the assay (both within the sample and the vessel holding the sample) that also fluoresce.

Often, fluorescence intensity assays will use multiple fluorophores to label different molecules in a single sample (e.g., cell, mixture or solution). This allows one to distinguish between these molecules based upon either the light used to excite the sample or the light emitted by the fluorophores. Another use of multiple fluorophore labels is fluorescence resonance energy transfer (“FRET”). FRET is based on the ability of one fluorophore to affect the fluorescence of another fluorophore in close proximity due to energy transfer between the fluorophores. In one use of FRET, the energy transfer between the fluorophores changes if the double-labeled molecule binds to another molecule, such as an antibody. Such binding prevents the energy transfer between the fluorophores [A. P. Wei et al., Anal. Chem., 66, pp. 1500-06 (1994)].

Another type of FRET utilizes excitation of a first donor fluorophore, which then emits at a wavelength that is absorbed by the second fluorophore if the two are within close enough proximity to one another. The second fluorophore then emits light at its emission wavelength. If the two fluorophores are not sufficiently close to one another or if the transfer of energy is otherwise blocked, light is emitted at the emission spectrum of the first fluorophore. The efficiency with which the energy transfer occurs is a function of the distance between the donor and the acceptor. FRET assays have been used to identify interactions between two molecules (one labeled with the donor and one with the acceptor), as well as the cleavage of a molecule (labeled with both the donor and the acceptor on different sides of the cleavage site). In the above types of FRET there is emission at one color (the emission wavelength of the first fluorophore) or another (the emission wavelength of the second fluorophore).

Another type of FRET uses one fluorophore and a quenching molecule that is not fluorescent, but absorbs light from the first fluorophore very efficiently. When the two are in close proximity, the excitation light is absorbed by the fluorophore and the quenching molecule absorbs its resulting emission light. When the two molecules are separated, the fluorophore emits light. In this type of FRET there is either no emission or one color emission.

Yet another method for utilizing fluorescently labeled molecules is fluorescence polarization (FP) (see J. C. Owicki, J. Biomolecular Screening, 5, pp. 297-306 for a review). FP is used to determine if a fluorescently labeled small molecule binds to a much larger binding molecule, such as an antibody, a nucleic acid sequence, an enzyme, a receptor or a binding protein with certain specificity (see, e.g., PCT publication WO 98/18956; PCT publication WO 98/18956; PCT publication WO 98/05962; U.S. Pat. No. 6,326,142; U.S. Pat. No. 6,100,039; and U.S. Pat. No. 6,202,397). FP has also been used to determine if a fluorescently labeled compound is degraded or digested; or if a fluorescently labeled molecule is incorporated into a larger molecule [e.g., see U.S. Pat. No. 5,786,139; T. M. Hsu et al., Biotechniques, 31, pp. 560-68 (2001); P. Y. Kwok, Hum. Mutat., 19, pp.315-23 (2002)]

FP is based upon the theory that when a fluorescently labeled molecule is excited with plane-polarized light of the correct wavelength it will emit polarized light after its characteristic emission lifetime (usually nanoseconds). During the time between excitation and emission (fluorescence lifetime), the molecule tumbles randomly with respect to the original plane of excitation. The rate of tumbling is directly proportional to its molecular volume or size. Small molecules tumble rapidly, while large molecules tumble much more slowly. If the molecule tumbles rapidly during its fluorescence lifetime, the fluorescence emission is depolarized relative to the excitation. If the labeled molecule is bound to a much bigger binding molecule, the complex is large, the tumbling decreases and the observed emission remains more polarized relative to the excitation. Thus by measuring the extent of fluorescence polarization, one can tell if a molecule is bound to a larger binding molecule. This technique can also be used to assay for competitors for binding to the binding molecule, which will displace the labeled ligand and decrease FP.

One advantage of FP is that it can be utilized in homogeneous assays, such as HTS. FP is also amenable to performing assays in real-time, directly in solution and without the need for an immobilized phase. Polarization values can be measured repeatedly and after the addition of reagents since measuring the samples is rapid and does not destroy the sample.

An FP assay adapted to detect single nucleotide polymorphisms and utilizing different probes labeled with different fluorophores has been recently described [T. M. Hsu et al., Clin.Chem., 47, pp. 1373-77 (2001)]. The advantage of this assay is that the binding of either probe to the nucleotide sequence target can be detected in a single tube or reaction well and distinguished based upon the excitation/emission wavelengths utilized.

FP does however, have some limitations in that interference with the assay can occur if other materials in the assay mix fluoresce, even weakly, at the wavelength that is used to perform FP [see J. C. Owicki, supra, for a detailed description of different types of interferences associated with FP]. This is because in most FP assays the test compound is present at a much higher concentration (100 to 10,000 fold higher) than the labeled compound. The use of a low concentration of labeled compound allows the assay to be much more sensitive and to distinguish between low, medium and high affinity binding by the test compound. It has been reported that interference by test compound fluorescence can cause up to 15% of a compound library to be detected as false positives or negatives when using fluorescein as the label. Numbers between 5 and 10% false positive compounds have been reported for assays employing tetramethylrhodamine as the label (Panvera customer's personal communication). A similar problem can occur when FP is used to detect oligomerization/deoligomerization in the presence of a test compound suspected of being an inhibitor of the enzyme responsible for oligomerization/deoligomerization.

Accordingly, there exists the need for ways to carry out FP to reduce or eliminate the problem of test compounds that fluoresce or otherwise interfere with FP measurements at particular wavelengths.

### SUMMARY OF THE INVENTION

Applicants have provided a method for performing FP that reduces the problems caused by interference from test compounds or other components that may be present in the sample to be assayed by FP. The term “test compound” as used herein means an unlabeled compound that is being evaluated either directly or indirectly in an FP assay. The invention provides a method for performing FP that utilizes multiple fluorophores on a compound and FP measurements at the excitation/emission wavelengths of each of the fluorophores. The method can be carried out using molecules that are each labeled with at least two fluorophores or using a mixture of molecules each of which is labeled with a different single fluorophore. Test compounds and other assay components that naturally fluoresce can interfere with the FP assay at only one of the excitation wavelengths. Thus a comparison of FP at multiple wavelengths will allow one to identify whether the fluorescence polarization observed is due to a real change in the binding of the labeled compound to a binding partner or is due to interfering fluorescence of the test compound. When test compounds fluoresce they interfere with polarization measurements such that the measurements are out of the range normally seen for the bound versus unbound and large versus small labeled compound. Another indication of the presence of interfering fluorescence is that the fluorescence intensity of the sample at the excitation/emission wavelength of a fluorophore is much different from the fluorophore alone or in the presence of a binding partner. In fluorescence polarization experiments, the emission total intensity remains constant even though the polarization value may change. Only the plane of the emission light changes between bound and unbound compound. Interfering compounds can either quench the emission intensity of the labeled compound or could be fluorescent by the same light as the labeled compound and increase the apparent emission intensity. Any significant intensity change will alter the polarization reading of the labeled compound, resulting in artificial results.

The invention also provides kits for carrying out the methods of this invention. The kits of this invention may contain a compound already labeled with one or more fluorophores or it may contain the fluorophores separate from the compound, thus requiring the user to label the compound before use.

### BRIEF DESCRIPTION OF THE DRAWINGS

FIG. 1, panel A, depicts the change in FP of a mixture of PKC antibody and either a fluorescein-labeled phosphopeptide (“Green”) or a fluorescein-labeled phosphopeptide and a tetramethylrhodamine-labeled phosphopeptide (“Green/Red”) caused by increasing concentration of an unlabeled phosphopeptide (“PKC Comp.”) as measured with a green filter. Panel B depicts the change in FP of a mixture of PKC antibody and either a tetramethylrhodamine-labeled phosphopeptide (“Red”) or a fluorescein-labeled phosphopeptide and a tetramethylrhodamine-labeled phosphopeptide (“Red/Green”) caused by increasing concentration of an unlabeled phosphopeptide (“PKC Comp.”) as measured with a red filter.

FIG. 2, panel A, depicts the change in FP of a mixture of PKC antibody, a fluorescein-labeled phosphopeptide and a tetramethylrhodamine-labeled phosphopeptide in the presence of increasing concentrations of an unlabeled phosphopeptide (“PKC Comp.”) alone (“Nothing”), or together with varying concentrations of free fluorescein (“FITC”) or free tetramethylrhodamine (“TAMRA”) as measured by a green filter. Panel B depicts the same experiment in panel A, except that FP was measured using a red filter.

FIG. 3, panel A, depicts the effect of varying concentrations of unlabeled dexamethasone on the FP of a mixture of soluble glucocorticoid receptor and (a) a steroid labeled with fluorescein (“Green”) or (b) a combination of a steroid labeled with fluorescein and a steroid labeled with tetramethylrhodamine each present at 0.5 nM (“0.5 & 0.5”) or at 0.8 nM (“0.8 & 0.8”) as measured with a green filter. Panel B, represents the same experiment in panel A, except that FP was measured using a red filter. Panel C depicts the effect of varying concentrations of unlabeled dexamethasone on the FP of a mixture of soluble glucocorticoid receptor and a combination of a steroid labeled with fluorescein and a steroid labeled with tetramethylrhodamine each present at 0.8 nM alone (“0.8 & 0.8”) or in the presence of 10 nM unconjugated fluorescein (“0.8 & 0.8+10 nMFITC”) or 5 nM tetramethylrhodamine (“0.8 & 0.8+5 nMTAMRA”) as measured with a green filter. Panel D depicts the same experiment as in Panel C, except that FP was measured with a red filter.

### DETAILED DESCRIPTION OF THE INVENTION

In one embodiment, the invention provides a method of determining if a population of compounds binds to a binding partner by fluorescence polarization, comprising the steps of:

a. providing a mixture comprising at least two subpopulations of compounds, wherein:

i. each subpopulation is characterized by a single compound labeled with a fluorophore;

ii. the fluorophore in any subpopulation has a maximal excitation peak that is at least 30 nanometers different from the maximal excitation peak of the fluorophore in another subpopulation and a maximal emission peak that is at least 30 nanometers different from the maximal emission peak of the fluorophore in another subpopulation;

iii. the labeled compound in each of the subpopulations has a dissociation constant for binding to said binding partner that is less than 50-fold different from the dissociation constant for the binding of the labeled compound in any other of the subpopulations; and

b. comparing the fluorescent polarization values of said mixture in the absence and the presence of said binding partner at multiple excitation and emission wavelengths that correspond to the wavelengths at which each of said fluorophores present in said mixture optimally absorbs and emits.

The term “population of compounds” as used herein means a sample characterized by two or more subsets of chemically related or identical molecules, wherein each subset is labeled with a different fluorophore. As such, each subset or “subpopulation” is homogeneous with respect to the chemical structure of the compounds and the fluorophore with which that compound is labeled.

The choice of fluorophore with which to label a subpopulation is based upon factors well known in the art, as well as the identity of fluorophores that are used to label other subpopulations within the population of compounds. One factor to be considered is the structure of the compound to be labeled, in particular the identity of moieties on the compound to which the fluorophore can be attached. Fluorophores are commercially available and often a fluorophore may be purchased with one of a variety of side groups, wherein different side groups are used to attach the fluorophore to different moieties. Thus, depending upon the structure of the compound to be labeled, one would choose the desired fluorophore containing the appropriate side group.

Another factor to be considered in the choice of fluorophore is that it should not sterically or chemically interfere with the ability of the compound to bind to its binding partner. Also, the fluorophore must demonstrate a significant difference in its FP in the bound versus free state. Both of these factors can be tested by simply labeling the compound, measuring its fluorescence polarization at the fluorophore's emission maxima, adding the binding partner under conditions that would allow the compound to bind to the binding partner, measuring the FP at the fluorophore's emission maxima and determining if and to what extent the FP has increased. To determine if the fluorescent label is interfering with the compound binding, a competition experiment can be performed to measure how the unlabeled compound and the labeled compound compete for binding to the partner.

According to this invention, the fluorophore used to label any given subpopulation of compound in the population must also have a maximal excitation peak that is at least 30 nanometers different from the maximal excitation peak of the fluorophore in another subpopulation in that population and a maximal emission peak that is at least 30 nanometers different from the emission excitation peak of the fluorophore in another subpopulation in that population. The basis behind the invention is that components present in the assay may cause interference because they either absorb or emit energy at the same wavelength as a fluorophore. By utilizing a second fluorophore that has a different maximal excitation and emission peak one can avoid such interference. We believe that a 30 nanometer or greater difference between the maximal excitation or emission peaks of the first and second fluorophores is sufficient to distinguish between the two fluorophores in a read out and to eliminate the interference caused by other components present in the assay when FP is measured using the excitation/emission wavelengths of one of the fluorophores.

Any different combination of fluorophores that meet these criteria may be utilized in this invention, as long as they have suitable biochemical and fluorescent properties and a detectable shift in polarization when bound versus free, as set forth above. Many dyes can be chosen from those listed below, but there are a few classes of dyes that cover a broad range of the spectrum. These include the xanthine dyes, the cyanine dyes, the Alexa dyes, the squarine dyes, and the bodipy dyes. Many other dyes can also be used but these listed below are commercially available and well characterized. The dyes are supplied with many different conjugation chemistries allowing them to be attached to a variety of moieties on the compound. Table 1, below, is a partial list of commercially available fluorophores that may be used in this invention.

TABLE 1
Commercially available fluorophores and their excitation
and emission maxima.

| Dye | Absorbance/Emission (nm) |
|---|---|
| AF 350 | 346/442 |
| AF 430 | 433/539 |
| AF 488 | 495/519 |
| AF 532 | 532/554 |
| AF 546 | 556/573 |
| AF 568 | 578/603 |
| AF 594 | 590/617 |
| AF 633 | 632/647 |
| AF 647 | 650/668 |
| AF 660 | 663/690 |
| AF 680 | 679/702 |
| Dintrophenyl | 349/NA |
| AMCA | 346/442 |
| Cascade Blue | 400/420 |
| Marina Blue | 365/460 |
| Fluorescein/FITC | 494/518 |
| Oregon Green 488 | 496/524 |
| Rhodamine Green | 505/527 |
| BODIPY FL | 504/513 |
| BODIPY TMR | 535/574 |
| BODIPY TR | 588/617 |
| Oregon Green 514 | 511/530 |
| Rhodamine Red | 570/590 |
| Tetramethylrhodamine | 555/580 |
| Texas Red | 595/615 |
| BODIIPY 630/650 | 632/640 |
| BODTPY 650/665 | 651/660 |
| QSY 7 | 560/NA |
| FluorX | 494/520 |
| Cy2 bis | 489/506 |
| Cy3 mono | 550/570 |
| Cy3.5 mono | 581/596 |
| Cy5 mono | 649/670 |
| Cy5.5 mono | 675/694 |
| Cy7 mono | 743/767 |
| DEAC | 432/472 |
| R6G | 524/552 |
| TAMRA | 547/573 |

According to one embodiment, one of said fluorophores is a green fluorophore having an emission maximum between 480 and 530 nm and another of said fluorophores is a red fluorophore having an emission maximum of greater than 570 nm. A more preferred embodiment is wherein the green fluorophore is fluorescein and the red fluorophore is either a tetramethylrhodamine dye or a cyanine dye.

Between subpopulations in the population of compounds the chemical structure of the compound may vary. In some embodiments, this allowed variability is beneficial in that the attachment of certain fluorophore may require moieties that need to be added to a compound. According to the invention it is necessary, however, that the ability of a compound in any given subpopulation to bind the binding partner, as measured by dissociation constant, be less than 50-fold different from the ability of a compound in any other subpopulation within the “population of compounds” to bind that binding partner. Those of skill in the art will realize that the variations in compounds will have to be relatively minor and located outside of the region responsible for binding to the binding partner in order to maintain the less than 50-fold difference in dissociation constant. Thus, changes such as the deletion, substitution or addition of amino acids or nucleotides in a peptide or oligonucleotide are envisioned, as long as those changes do not involve the binding domain and do not cause a significant change in three-dimensional structure. For small molecules, we envision changes such as the deletion, substitution or addition of small moieties (e.g., methyl groups, hydrogen/halogen substitutions, addition of ring substituents, etc.) that do not involve the portion of the molecule that binds to the binding partner. For some compounds, different chemical linkers that are used to attach the label to the compound can positively or negatively alter the compound binding to the binding partner. This is to be expected, but as long as the binding affinities remain within 50-fold, the compounds may be used together in this invention to form the population of compounds used in the binding assay.

One may easily determine the dissociation constant for each of the compounds present in the population using standard assays employing the individual compound and the binding partner. In a typical experiment, the binding partner is serially diluted 2-fold in a binding buffer over a ten thousand fold range, in duplicate. The dilution concentrations should span the expected dissociation constant by 100 fold. One compound is added to each diluted series at a concentration that is at least three fold below the expected dissociation constant. After allowing the binding reactions to reach equilibrium (typically a few minutes up to a couple of hours), the fluorescence polarization value is measured. The dissociation constant is defined as the binding partner concentration where one-half of the labeled compound is bound. If the calculated dissociation constant is within 3-fold of the dissociation constant, the experiment can be repeated using a lower concentration of labeled compound.

The term “binding partner” as used herein refers to any molecule that is capable of binding all of the compounds within the population. These include, but are not limited to, an enzyme, receptor, antibody, other protein, carbohydrate, lipid, or nucleic acid sequence. Examples of binding partners include, but are not limited to, a polyclonal or monoclonal antibody or antigen binding portion thereof wherein each of the compounds in the population are antigens that are bound by the antibody; an SH2 domain-containing molecule where the compounds in the population contain a phosphorylated amino acid; a nuclear receptor or binding portion thereof where the compounds in the population are steroids or molecules which are not chemically defined as steroids, but mimic steroid binding and/or function (“steroid mimics”) that bind to said receptor; a cell surface receptor or binding portion thereof, wherein the compounds in the population are ligands for the particular receptor; a single-stranded nucleic acid sequence wherein the compounds in the population are short oligonucleotides that are capable of hydrogen bonding with that nucleotide sequence due to sufficient complimentarity; nucleic acid binding proteins where the compounds in the population are nucleic acid sequences which contain the recognition sequence for the nucleic acid binding protein; a nuclear receptor protein where the compounds are peptides or small protein domains that recognize the protein regulatory sequence with the nuclear receptor; and a lectin molecule where the compound is a sugar which specifically recognizes the lectin.

It will be understood by those of skill in the art that the after the binding partner is added to the labeled compound, one must allow time sufficient for the compound to bind to the binding partner before FP measurements are taken. The amount of time necessary to allow binding to occur will depend on the nature of the compound and the binding partner and the temperature of the solution containing the binding partner and the compound. Typically, binding is allowed to occur over a period from about 15 minutes to 6 hours, more preferably over a period of 1 to 3 hours.

The last step of this method involves multiple FP measurements, both in the absence and presence of the binding partner, of the multiple fluorophore-labeled population of compounds. The FP measurements are taken by exciting the population at a wavelength that corresponds to the optimal absorption (“excitation”) wavelength of one fluorophore and reading the resultant polarization at a wavelength that corresponds to the optimal emission wavelength of that fluorophore. The process is repeated using wavelengths that correspond to the optimal excitation and emission wavelengths for each of the fluorophores present in the population. If there is no interference from the assay components, the final polarization value should be between the high polarization of the compound/binding partner complex and the low polarization of the unbound compound for each FP measurement. Also, the total intensity in the assay should not change significantly when the assay component is added. Excitation and emission spectra, as well as optimal wavelengths for carrying out fluorescence studies are well known for individual fluorophores and are typically available from the commercial source of the fluorophores. It will be readily apparent that the apparatus used to measure FP is equipped with a finite number of filters. Accordingly, the choice of filter to use for excitation and for emission will be chosen to be as close as possible to the excitation/emission optima for the given fluorophore to be measured.

According to another embodiment, the invention provides a method of determining if a compound binds to a binding partner by fluorescence polarization, comprising the steps of:

a. providing a compound labeled with at least two different fluorophores, wherein:

i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound, and

ii. there is less than 50% fluorescence resonance energy transfer between any two of the fluorophores present in the labeled compound; and

b. comparing the fluorescent polarization values of said compound in the absence and the presence of said binding partner at multiple wavelengths that correspond to the wavelengths at which each of said fluorophores present on said compound optimally absorbs and emits.

This method differs slightly from the method described above in that the compound utilized in the FP assay is labeled with at least two fluorophores. Similar to the embodiment described above, each of the two or more fluorophores has a maximal emission peak that is at least 30 nanometers different from the maximal emission peak of another fluorophore on said compound and a maximal excitation peak that is at least 30 nanometers different from the maximal excitation peak of another fluorophore on said compound.

In addition, when compounds are labeled with two or more fluorophores there exists the possibility of fluorescence resonance energy transfer (“FRET”) between the two fluorophores. FRET is desirable in some fluorescence-based assays and is purposely utilized to determine if the distance between the two fluorophores has changed (e.g., if the molecule containing the two fluorophores has been cleaved into two pieces, each containing one of the fluorophores). However, in the methods of the present invention, FRET should be avoided as it tends to reduce the sensitivity of the assay or worse, eliminates emission at the expected wavelength, thus making FP measurements difficult if not impossible.

Thus, according to this embodiment of the invention, there must be less than 50% FRET between any two of the fluorophores present in the labeled compound. Preferably there will be less than 30% FRET and more preferably less than 5% FRET between any two of the fluorophores present in the labeled compound. The determination of whether or not two fluorophores will exhibit FRET is based upon the degree of which the emission spectrum of one fluorophore overlaps the excitation spectrum of another fluorophore, the distance between the two fluorophores on the compound, and the degree to which the transition dipole orientations of the two fluorophores are parallel.

The effect of distance between the fluorophores on FRET is based upon the Förster radius (Rₒ), which represents the distance at which energy transfer is 50% efficient (i.e., 50% of excited donors are deactivated by FRET). The magnitude of Rₒ is dependent on the spectral properties of the donor and acceptor dyes and is calculated by the formula:

Rₒ=[8.8×10²³·κ²·n⁻⁴·QY_{D}·J(λ)]^{⅙}Å

where κ²=dipole orientation factor (range 0 to 4; κ²=⅔for randomly oriented donors and acceptors)

QY_{D}=fluorescence quantum yield of the donor in the absence of the acceptor

n=refractive index

J(λ)=spectral overlap integral (see figure) =∫ε_{A}(λ)·F_{D}(λ)·λ⁴dλcm³M⁻¹

where ε_{A}=extinction coefficient of acceptor

F_{D}=fluorescence emission intensity of donor as a fraction of the total integrated intensity

A listing of the Forster radius for various dyes is typically available from the manufacturer (see, for example, http://www.molecularprobes.com/handbook/boxes/0422.html). Thus, one may determine theoretically whether two fluorophores will exhibit less than 30% FRET based upon their relative location on the compound. Two fluorophores that may exhibit FRET due to overlap between the emission spectrum of one and the excitation spectrum of the other must be separated by greater than the Forster radius of either fluorophore.

As a general rule, the larger the compound to be labeled, the easier it will be to avoid FRET simply by placing sufficient distance between the two fluorophores. Thus when the compound to be labeled is a peptide or an oligonucleotide, there is less chance of overlapping emission/excitation spectra since these types of molecules are large enough to space the fluorophores at distances greater than their Forster radius. For compounds that are small molecules, there is less of an opportunity to sufficiently space the fluorophores to avoid FRET. Accordingly, one must utilize fluorophores where the emission spectrum of the fluorophore excited at the shorter wavelength is sufficiently different from the excitation spectrum of the other fluorophore on the molecule to avoid more than 50% FRET.

Once fluorophores have been selected, one may determine the degree of FRET exhibited by those two fluorophores when present on a compound by a simple experimental technique. A subpopulation of compound is labeled with a first fluorophore. Another subpopulation of the same compound is labeled with the first fluorophore and a second fluorophore. The fluorescence intensity of the singly labeled compound is measured using the appropriate excitation/emission wavelengths of light. This is then compared to the fluorescence intensity of the double-labeled compound at the same wavelengths. The decrease in fluorescence intensity of the latter compound represents FRET and must be less than 50% in order to use the doubly-labeled compound in the methods of this invention.

In this embodiment, the final step is identical to the prior embodiment—taking multiple FP measurements, both in the absence and presence of the binding partner, of the multiple fluorophore-labeled compound. The FP measurements are taken by exciting the compound at a wavelength that corresponds to the optimal excitation wavelength of one fluorophore and reading the resultant polarization at a wavelength that corresponds to the optimal emission wavelength of that fluorophore. The process is repeated using wavelengths that correspond to the optimal excitation and emission wavelengths for each of the fluorophores present on the compound.

Labeling a compound with multiple fluorophores at different sites is achieved by methods well known in the art. The choice of fluorophores will be based upon the factors enumerated above, as well as the compatibility of the reactive groups on the fluorophore with the compound to be labeled. According to one embodiment, two or more fluorophores are conjugated to one another when present on the compound. The conjugation of fluorophores to one another may be achieved by chemistry well-known in the art. For example, many commercially available fluorophores have multiple distinct reactive groups, one of which may be used to couple the fluorophore to another fluorophore and another that may be used to couple the conjugate to the compound. It will be readily apparent to those of skill in the art that successful conjugation of two fluorophores requires compatible reactive groups on each fluorophore to allow them to be coupled to one another.

Similarly, appropriate reactive groups can be created, if they do not exist in the commercially obtained fluorophores, by using simple chemical reaction to alter existing reactive moieties on the fluorophore. For example, 4,4-difluoro-4-bora-3a,4a-diaza-s-indacene-3,5-dipropionic acid (BODIPY® 500/510 excitation/emission wavelengths; Molecular Probes, Eugene, Oreg. Catalog #D-6103):

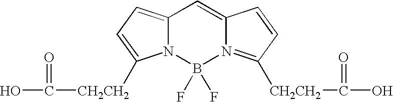

could be used to prepare the water-soluble amine reactive sulfosuccinimidyl esters which can then be coupled to a reactive amine-containing fluorophore.

The order in which the fluorophores are coupled to one another and to the compound may vary depending upon the nature of the reactive groups present in the fluorophore and the compound. Since the compound is likely to be the most chemically complex component, it will usually be preferred to conjugate the two fluorophores together and then react that conjugate with the compound.

The methods of the present invention are compatible with any known use of FP that involves the use of a fluorescently labeled compound and a binding partner for that compound. These uses include enzyme assays wherein the substrate is the labeled compound (PCT publication WO 98/18956), enzyme assays wherein the enzyme product competes with the labeled compound for binding to the binding partner (PCT publication WO 98/18956), receptor binding assays wherein the labeled compound is a ligand for the receptor (PCT publication WO 98/05962), assays to quantitate dissociation constants between a labeled oligonucleotide and a protein or nucleic acid (U.S. Pat. No. 6,326,142), assays to measure quantity of DNA produced by reverse transcriptase, wherein a labeled oligonucleotide probe is bound to the single-stranded DNA produced by the reverse transcriptase (U.S. Pat. No. 6,100,039) and assays to determine the association between a labeled ligand and a molecule containing an SH2 domain, an SH3 domain, or a phosphotyrosine interaction domain (U.S. Pat. No. 6,202,397). The disclosures of each of these patents and patent publications are hereby incorporated by reference.

According to one embodiment, the fluorescence polarization values taken in the presence of the binding partner are determined in the presence of a test compound. As stated above, a major contributor to fluorescence polarization interference is often a test compound that inherently fluoresces at a wavelength that overlaps the excitation/emission wavelength of a fluorophore present on the compound or population of compounds. Thus the multi-fluorophore labels present on the compound or population of compound allows one to obtain an FP measurement at a non-interfering wavelength.

In a first preferred embodiment, the test compound is a substance suspected of being a modulator of the ability of said binding partner to bind to said compounds or compound. This embodiment is directed to assays that probe a test compound's ability to compete with the fluorophore-labeled compound or compounds for binding to the binding partner; to enhance the binding of the fluorophore-labeled compound or compounds for binding to the binding partner; to bind to the binding partner at a different site and cause a conformational change in the binding partner so that it can no longer bind the labeled compound(s); to bind to the binding partner at a different site and cause a conformational change in the binding partner so that it has greater affinity for the labeled compound(s); to otherwise affect a binding partner so that it can not bind to the labeled compound; to otherwise affect a binding partner so that it binds with greater affinity to the labeled compound; to bind to the labeled compound and prevent the latter from binding to the binding partner; to bind to the labeled compound and enhance its ability to bind to the binding partner; to otherwise interact with the labeled compound, causing a change in the latter so that it can no longer bind the binding partner; or to otherwise interact with the labeled compound, causing a change in the latter so that it has greater affinity for binding to the binding partner. One example of such an assay is wherein the binding partner is a receptor, the labeled compound(s) is a ligand for said receptor and the test compound is suspected of being a competitive inhibitor of said ligand for binding to the receptor. Other examples of such assays are well known in the art and are set forth in the specific examples.

In a second preferred embodiment, the test compound is a substance that is suspected of being a modulator of a molecule that inhibits the ability of said binding partner to bind to said labeled compound or compounds. This embodiment is directed to assays that probe, for example, a test compound's ability to modulate an enzyme for which the labeled compound(s) is a substrate or for which the binding partner is a substrate (wherein, in either case, the enzymes action on the substrate alters the ability of that substrate to bind to the other component). It is also directed to assays that look at a test compound's ability to modulate a molecule that has an affinity for binding to either the binding partner or the labeled compound(s). In this embodiment, the test compound must be allowed to contact the molecule for a period of time and under conditions that would allow a modulator to affect said molecule. This may occur in the presence of the labeled compound and/or binding partner. Alternatively, the test compound may be contacted with molecule prior to being added to the labeled compound and the binding partner. One example of such an assay is wherein the molecule is a phosphatase, the test compound is a suspected inhibitor of said phosphatase, the labeled compound is a phosphorylated peptide and the binding partner is a phosphate-binding molecule, such as an antibody specific for a phosphorylated amino acid present in the peptide or a protein containing an SH2 domain. Other examples of such assays are well known in the art and are set forth in the specific examples.

In a third preferred embodiment, the test compound is a substance that is suspected of being a modulator of an enzyme that acts upon a substrate to produce a product that interferes with the ability of the labeled compound(s) to bind to the binding partner. In this embodiment, it is the product of the enzymatic reaction that may either compete for binding to the binding partner or otherwise affect the binding partner or labeled compound(s) in such a manner as to impede its ability to bind to the other component. In this embodiment, the test compound must be incubated with the enzyme and the substrate for a period of time and under conditions that would allow said enzyme to act upon said substrate in the absence of a modulator. As in the previous embodiment, this may occur in the presence of the labeled compound and/or binding partner or prior to being added to the labeled compound and the binding partner. One example of such an assay is where the enzyme is a kinase, the substrate is phosphorylatable peptide, the test compound is a potential inhibitor of the kinase, the labeled compound is a phosphorylated peptide and the binding partner is a phosphate-binding molecule, such as an antibody specific for a phosphorylated amino acid present in the peptide or a protein containing an SH2 domain. Other examples of such assays are well known in the art and are set forth in the specific examples.

In one embodiment, the methods of the present invention utilize a phosphopeptide or a population of phosphopeptides as the labeled compound(s) and a phosphate-binding molecule as the binding partner. Suitable binding partners in this embodiment include, antibodies specific for phosphorylated amino acid-containing antigens, molecules containing an SH2 domain, molecules containing a 14-3-3 domain, or phosphate binding moieties such as metal ions. In another embodiment, the methods of the present invention utilize a steroid hormone or steroid hormone mimic or a population of steroid hormones, steroid hormone mimics or combinations thereof, as the labeled compound or population of compound(s) and a nuclear receptor as the binding partner. In yet another embodiment, an oligonucleotide or population of oligonucleotides is the labeled compound or population of compounds and the binding partner is either a nucleic acid binding protein or a nucleic acid sequence containing a sequence of nucleotides that is complimentary to the labeled oligonucleotide(s)

These methods are particularly useful in identifying potential inhibitors of enzymes that may be linked to diseases. For example, kinases are known to play roles in cancer, diabetes, heart disease and many other maladies. Kinase inhibitors identified by these methods are potential therapeutics for treating and preventing those diseases that are specific to the kinase being assayed.

According to another aspect of the invention, there is provided a method of determining by fluorescence polarization if a population of compounds is incorporated into a larger molecule. This method comprises the steps of:

a. providing a first mixture comprising at least two subpopulations of compounds, wherein:

i. each subpopulation is characterized by a single compound labeled with a fluorophore;

ii. the fluorophore in any subpopulation has a maximal excitation peak that is at least 30 nanometers different from the maximal excitation peak of the fluorophore in another subpopulation and a maximal emission peak that is at least 30 nanometers different from the maximal emission peak of the fluorophore in another subpopulation;

iii. the labeled compound in each of the subpopulations is capable of being incorporated into a larger molecule through the action of an enzyme or a catalyst; and

iv. the labeled compound in each of the subpopulations has a rate of incorporation into a larger molecule through the action of said enzyme or catalyst that is less than 50-fold different from the rate of incorporation into a larger molecule by said enzyme or catalyst of the labeled compound in any other of the subpopulations;

b. measuring the fluorescence polarization values of said first mixture at multiple wavelengths that correspond to the wavelengths at which each of said fluorophores present in said first mixture optimally absorbs and emits;

c. adding to said first mixture a solution comprising said enzyme or catalyst capable of incorporating said compounds into a larger molecule to create a second mixture, wherein said second mixture is incubated for a period of time and provides conditions that, in the absence of an inhibitor of said enzyme or catalyst, allow said compounds to be incorporated into a larger molecule through the action of said enzyme or catalyst;

d. measuring the fluorescence polarization values of said second mixture at multiple wavelengths that correspond to the wavelengths at which each of said fluorophores present in said mixture optimally absorbs and emits;

e. comparing the fluorescence polarization measurements taken at steps b and d to determine if said population of compounds have been incorporated into a larger molecule.

In this embodiment, the parameters for choosing fluorophores and for labeling subpopulations of compounds with different fluorophores is identical to those set forth above for assays that utilize binding partners.

In this aspect of the invention, the labeled population of compounds are relatively small “building block” components that are normally incorporated into larger molecules through the action of an enzyme or a catalyst. These include, for example, amino acids, nucleotides, simple sugars, and other small organic molecules or radicals that are subject to polymerization. The FP of the labeled compounds alone would be low. Once the labeled compounds are incorporated into a larger molecule, the FP would increase.

The choice of enzyme or catalyst utilized in this embodiment will of course be based upon the nature of the labeled compound(s). Thus, if the labeled compound(s) are amino acids, the enzyme would be selected from, for example, a transacetylase or an amidotransferase. If the labeled compound(s) are deoxyribonucleotides, the enzyme would be selected from, for example, a DNA polymerase, or a reverse transcriptase. If the labeled compound(s) are ribonucleotides, the enzyme would, for example, be an RNA polymerase. If the labeled compound(s) are other polymerizable small organic molecules or radicals, the enzyme would be, for example, a polymerase or the catalyst would be an appropriate metal ion.

As in the embodiments that utilize a binding partner described above, the chemical structure of the compound may vary between subpopulations in the population of compounds the. According to this embodiment is necessary that the ability of a compound in any given subpopulation to be incorporated into a larger molecule by the action of an enzyme or catalyst be less than 50-fold different from the ability of a compound in any other subpopulation within the “population of compounds” to be incorporated into a larger molecule by the action of the same enzyme or catalyst.

Those of skill in the art will realize that the variations in compounds will have to be relatively minor and in order to maintain the less than 50-fold difference in rate of incorporation. Thus, changes such as the deletion, substitution or addition of amino acids or nucleotides in a peptide or oligonucleotide are envisioned, as long as those changes do not cause a significant change in the ability of the enzyme or catalyst to recognize the compound.

The conditions that allow said labeled compound(s) to be incorporated into a larger molecule through the action of said enzyme or catalyst will vary according to the nature of the enzyme and the labeled compound. Optimal conditions for various enzymes and catalysts envisioned by this embodiment are well known in the art and usually include a narrow buffered pH range, the possible presence of certain ions or other co-factors, the possible presence of NaCl or other salts, and an appropriate temperature. The amount of time necessary for incorporation of the labeled compound(s) into a larger molecule is also variable dependent upon the nature of the compound and the enzyme or catalyst. Typically, such reactions should be allowed to proceed for 15 minutes to 24 hours. FP measurements can be taken once the reaction is terminated or at multiple times during the course of the reaction.

In an alternate embodiment, the invention provides a method of determining by fluorescence polarization if a compound is incorporated into a larger molecule, comprising the steps of:

a. providing a first solution comprising a compound labeled with at least two different fluorophores, wherein:

i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound;

ii. there is less than 50% fluorescence resonance energy transfer between any two of the fluorophores present in the labeled compound; and

iii. said compound is capable of being incorporated into a larger molecule through the action of an enzyme or a catalyst;

b. measuring the fluorescence polarization values of said first solution at multiple wavelengths that correspond to the wavelengths at which each of said fluorophores present on said compound optimally absorbs and emits;

c. adding to said first solution a solution comprising said enzyme or catalyst capable of incorporating said compounds into a larger molecule to create a second solution, wherein said second mixture is incubated for a period of time and provides conditions that, in the absence of an inhibitor of said enzyme or catalyst, allow said compound to be incorporated into a larger molecule through the action of said enzyme or catalyst;

d. measuring the fluorescence polarization values of said second solution at multiple wavelengths that correspond to the wavelengths at which each of said fluorophores present in said mixture optimally absorbs and emits; and

e. comparing the fluorescence polarization measurements taken at steps b and d to determine if said compound has been incorporated into a larger molecule.

This embodiment is similar to the one set forth previously except that a single compound labeled with multiple fluorophores is used. The choice of and relationship between fluorophores and the parameters and methods for labeling a compound with multiple fluorophores, including the preferred embodiments, are identical to that described above for assays employing a binding partner.

In a preferred embodiment of both methods, a substance suspected of being a modulator of the ability of said enzyme or said catalyst to incorporate said labeled compound into a larger molecule is present prior to step d. The FP measurements taken at step d are in the presence of this substance. Typically, this substance would be suspected of being a modulator of the enzyme or catalyst. However, substances that act upon the labeled compound to modulate its rate of being incorporated into a larger molecule are also envisioned. The substance may be present prior to the addition of the enzyme or catalyst or may be added together with the enzyme or catalyst. If the substance is suspected of being a modulator of the enzyme or catalyst it should be contacted with said enzyme or catalyst for a period of time and under conditions that would allow a modulator to affect said enzyme or catalyst. This should be carried out prior to adding the enzyme or catalyst and the substance to the labeled compound. If the substance is suspected of interacting with the labeled compound, it should be contacted with the labeled compound for a period of time and under conditions that would allow such interaction. This should occur prior to adding the enzyme or catalyst to the labeled compound and the substance.

These methods are particularly useful in identifying potential inhibitors of viral polymerases, such as human immunodeficiency virus (“HIV polymerase”), hepatitis C virus (“HCV”) polymerase, hepatitis B virus polymerase (“HBV polymerase”), bacterial DNA polymerases, or bacterial transacetylases. Such inhibitors can disrupt the ability of the virus or bacteria to propagate and thus are candidates for therapeutic agents for the treatment or prevention of disease caused by these organisms.

In another aspect, the invention provides a method of determining by fluorescence polarization if a population of compounds is degraded into smaller molecules, said method comprising the steps of:

a. providing a first mixture comprising at least two subpopulations of compounds, wherein:

i. each subpopulation is characterized by a single compound labeled with a fluorophore;

ii. the fluorophore in any subpopulation has a maximal excitation peak that is at least 30 nanometers different from the maximal excitation peak of the fluorophore in another subpopulation and a maximal emission peak that is at least 30 nanometers different from the maximal emission peak of the fluorophore in another subpopulation;

iii. the labeled compound in each of the subpopulations is capable of being degraded into smaller molecules through the action of an enzyme; and

iv. the labeled compound in each of the subpopulations has a rate of degradation by said enzyme that is less than 50-fold different from the rate of degradation by said enzyme of the labeled compound in any other of the subpopulations;

b. measuring the fluorescence polarization values of said first mixture at multiple wavelengths that correspond to the wavelengths at which each of said fluorophores present in said first mixture optimally absorbs and emits;

c. adding to said first mixture a solution comprising said enzyme capable of degrading said compounds into smaller molecules to create a second mixture, wherein said second mixture is incubated for a period of time and provides conditions that, in the absence of an inhibitor of said enzyme, allow said compounds to be degraded into smaller molecules through the action of said enzyme;

d. measuring the fluorescence polarization values of said second mixture at multiple wavelengths that correspond to the wavelengths at which each of said fluorophores present in said mixture optimally absorbs and emits; and

e. comparing the fluorescence polarization measurements taken at steps b and d to determine if said population of compounds have been degraded into smaller molecules.

In this embodiment, the labeled compounds are large molecules, such as polypeptides, proteins, polynucleotides such as single-and double stranded DNA molecules and RNA molecules or organic polymers, each of which would have a high FP. If these molecules are degraded by an appropriate enzyme, such as a protease or a proteinase, for example, for a polypeptide or a protein; a DNase, exonuclease or an endonuclease, for example, for a DNA molecule; an RNase, for example for an RNA molecule or a hydrolase for an organic polymer, the resulting labeled products are much smaller compounds. These smaller labeled compounds have a much lower FP.

As with the previous embodiments, the choice of enzyme to use will be based upon the nature of the labeled compound(s). If the labeled compound is a protein or polypeptide the enzyme should be, for example, a protease or a proteinase. If the labeled compound is a DNA molecule the enzyme should be, for example, a DNase, exonuclease or an endonuclease. If the labeled compound is an RNA molecule, the appropriate enzyme will be, for example, an RNase. If the labeled compound is an organic polymer, the appropriate enzyme will be, for example, hydrolase.

The parameters for differences between compounds in each of the subpopulation that will not cause a greater than 50-fold difference in the rate of degradation are identical to those set forth above for compounds having less than 50-fold difference in their rates of incorporation into larger molecules.

The choice of fluorophores with which to label the population of compounds and the parameters and methods for achieving such labeling are identical to those set forth above, including the preferred embodiments for such choices, parameters and methods. Conditions that allow the enzyme to degrade the labeled compound will vary depending upon the enzyme used and are well known in the art. These conditions include a buffered pH, the potential presence of NaCl or other salts, the potential presence of metal ions or other cofactors for the enzyme and an appropriate temperature for maximal enzyme activity.

The amount of time necessary for degradation of the labeled compound(s) into smaller molecules is also variable dependent upon the nature of that compound and the enzyme. Typically, such reactions should be allowed to proceed for 15 minutes to 24 hours. FP measurements can be taken once the reaction is terminated or at multiple times during the course of the reaction.

According to an alternate embodiment, the invention provides a method of determining by fluorescence polarization if a compound is degraded into smaller molecules, said method comprising the steps of:

a. providing a first solution comprising a compound labeled with at least two different fluorophores, wherein:

i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound;

ii. there is less than 50% fluorescence resonance energy transfer between any two of the fluorophores present in the labeled compound; and

iii. said compound is capable of being degraded into smaller molecules through the action of an enzyme;

b. measuring the fluorescence polarization values of said first solution at multiple wavelengths that correspond to the wavelengths at which each of said fluorophores present on said compound optimally absorbs and emits;

c. adding to said first solution a solution comprising said enzyme capable of degrading said compound into smaller molecules to create a second solution, wherein said second mixture is incubated for a period of time and provides conditions that, in the absence of an inhibitor of said enzyme, allow said compound to be degraded into smaller molecules through the action of said enzyme;

d. measuring the fluorescence polarization values of said second solution at multiple wavelengths that correspond to the wavelengths at which each of said fluorophores present in said mixture optimally absorbs and emits; and

e. comparing the fluorescence polarization measurements taken at steps b and d to determine if said compound has been degraded into smaller molecules.

This embodiment is similar to the one set forth immediately above except that a single compound labeled with multiple fluorophores is used. The choice of and relationship between fluorophores and the parameters and methods for labeling a compound with multiple fluorophores, including the preferred embodiments, are identical to that described above for assays employing a multiple fluorophore labeled compound.

In a preferred embodiment of both methods, a substance suspected of being a modulator of the ability of said enzyme to degrade said labeled compound is present prior to step d. The FP measurements taken at step d are in the presence of said substance. Typically, this substance would be suspected of being a modulator of the enzyme. However, substances that act upon the labeled compound to alter its rate of being degraded are also envisioned. The substance may be present prior to the addition of the enzyme or may be added together with the enzyme. If the substance is suspected of being a modulator of the enzyme it should be contacted with said enzyme for a period of time and under conditions that would allow a modulator to affect said enzyme. This should be carried out prior to adding the enzyme and the substance to the labeled compound. If the substance is suspected of interacting with the labeled compound, it should be contacted with the labeled compound for a period of time and under conditions that would allow such interaction. This should occur prior to adding the enzyme to the labeled compound and the substance.

In each of the above embodiments employing a “test compound” (e.g. a substance suspected of some sort of inhibitory activity) and requiring pre-incubation of the test compound with one of the assay components it is possible to remove the test compound (as well an any other components that do not directly affect the FP measurements) prior to an FP measurement. However, such manipulations, although within the scope of the invention, are not preferred. This is because too many additional steps are required, particularly if the assay is designed to be high throughput or ultra-high throughput. Moreover, the very nature of fluorescence polarization and the use of multi-fluorophore labeled compound(s) as set forth herein is intended to avoid the need for any separation steps prior to making FP measurements. That is why this technique is so amenable to high throughput screening.

Methodology for carrying out each of the steps in all of the above methods, other than steps for labeling a compound with more than one fluorophore, is set forth in “Fluorescence Polarization. Technical Resource Guide, Third Edition”, available from PanVera Corporation, Madison, Wis., USA, the disclosure of which is herein incorporated by reference.

The present invention also provides kits for use in the methods described above. In one embodiment, a kit of this invention comprises:

a. a first subpopulation of a compound labeled with a first fluorophore;

b. a second subpopulation of a compound labeled with a second fluorophore that has a maximal excitation peak that is at least 30 nanometers different from the maximal excitation peak of the fluorophore in said first subpopulation and a maximal emission peak that is at least 30 nanometers different from the maximal emission peak of the fluorophore in said first subpopulation, wherein said compound in said second subpopulation has:

i. a dissociation constant for binding to a binding partner that is less than 50-fold different from the dissociation constant for the binding to said binding partner by said compound in said first subpopulation, or

ii. a rate of incorporation into a larger molecule by an enzyme or a catalyst that is less than 50-fold different from the rate of incorporation into a larger molecule by said enzyme or said catalyst of said compound in said first subpopulation, or

iii. a rate of degradation by an enzyme that is less than 50-fold different from the rate of degradation by said enzyme of said compound in said first subpopulation; and

c. instructions for using said kit which instruct the user to perform fluorescence polarization measurements on a mixture comprising said first and said second subpopulations of compound at multiple wavelengths corresponding to the wavelengths at which each of said first and second fluorophores emits.

In this embodiment, the subpopulation of compounds may be in separate vessels or, more preferably, they may be pre-mixed in a single vessel. The compounds in each subpopulation should be closely related chemically and with respect to their ability to interact with a binding partner or an enzyme. It is preferred if the subpopulation of compounds are chemically identical except for the identity of the different fluorophore label.

In one embodiment, the kit described above additionally comprises either:

a. an enzyme or catalyst capable of incorporating said compound in said first subpopulation and said compound in said second subpopulation into a larger molecule, wherein said compound in said second subpopulation has a rate of incorporation into a larger molecule by said enzyme or said catalyst that is less than 50-fold different from the rate of incorporation into a larger molecule of said compound in said first subpopulation by said enzyme or said catalyst, or

b. an enzyme capable of degrading said compound in said first subpopulation and said compound in said second subpopulation into smaller molecules, wherein said compound in said second subpopulation has a rate of degradation by said enzyme that is less than 50-fold different from the rate of degradation by said enzyme of said compound in said first subpopulation, or

c. a binding partner, wherein said compound in said second subpopulation has a dissociation constant for binding to said binding partner that is less than 50-fold different from the dissociation constant for the binding to said binding partner by said compound in said first subpopulation.

If the additional component is a catalyst or either of the two enzyme types described above, the enzyme should be present in a separate vessel from the labeled compounds. If the additional kit component is a binding partner, the binding partner may be present in a separate vessel or in the same vessel as one of the subpopulations of compounds or in the same vessel as the pre-mixed subpopulations of compounds.

According to another embodiment, the invention provides a kit comprising:

a. a compound labeled with at least two fluorophores, wherein:

i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound, and

ii. there is less than 50% fluorescence resonance energy transfer between any two of the fluorophores present in the labeled compound; and

b. instructions for using said kit which instruct the user to perform fluorescence polarization measurements on a solution comprising said compound at multiple wavelengths corresponding to the wavelengths at which each of said fluorophores emits.

According to yet another embodiment, the invention provides a kit comprising:

a. a compound;

b. a first fluorophore;

c. a second fluorophore that has a maximal emission peak that is at least 30 nanometers different from the maximal absorbance peak of the first fluorophore, wherein there is less than 50% energy transfer between said first and said second fluorophores when said compound is labeled with said fluorophores; and

d. instructions for using said kit which instruct the user to:

i. label said compound with said first and said second fluorophore; and

ii. perform fluorescence polarization measurements on a solution comprising said labeled compound at multiple wavelengths corresponding to the wavelengths at which each of said fluorophores emits.

In the kit containing the fluorophores separate from the compound, the fluorophores may be in separate vessels or combined in a single vessel. When they are combined in a single vessel, they may also be conjugated to one another. Techniques for conjugating two fluorophores are described above. Similarly in the kit containing the compound labeled with two fluorophores, the fluorophores may also be conjugated to one another on the compound.

In a preferred embodiment of either of these two kits, the kit additionally comprises:

a. a binding partner capable of binding said compound, or

b. an enzyme capable of incorporating said compound into a larger molecule, or

c. an enzyme capable of degrading said compound into smaller molecules.

When the binding partner is the additional component in a kit containing a multiple-labeled compound, the binding partner may be pre-mixed with the compound in the kit.

In kits containing a single compound (either unlabeled or labeled with two fluorophores), it is preferred that once the compound is labeled, that the two fluorophores exhibit less than 30% FRET, and more preferably, that they exhibit less than 5% FRET.

According to one embodiment, in each of the kits described above, the first fluorophore is fluorescein and the second fluorophore is a tetramethylrhodamine dye or a cyanine dye.

In order that this invention be more fully understood, the following examples are set forth. These examples are for the purpose of illustration only and are not to be construed as limiting the scope of the invention in any way.

### EXAMPLE 1

### Detection of Phosphorylated Peptide by Dual Color Fluorescence Polarization

An unlabeled phosphopeptide was serially diluted in 100 mM potassium phosphate pH 7.4, 0.1 mg/ml acetylated Bovine Gamma Globulin (BGG) buffer. This synthetic phosphopeptide was used as a competitor for binding to an antibody that also recognizes two different fluorophore-labeled phosphopeptides used in the experiment. The diluted competitor was incubated for 3 hours at room temperature with 6 nM anti-phosphopeptide antibody, 0.5 nM of the same phosphopeptide labeled with fluorescein, and 0.5 nM of the same phosphopeptide labeled with tetramethylrhodamine (TMR)-labeled. Two separate fluorescence polarization values were obtained for each sample. We obtained the first measurement by exciting the samples at 485 nm (green filters) and detecting the emitted fluorescence at 535 nm in a microwell plate in a fluorimeter capable of measuring fluorescence polarization. We obtained the second measurement by exciting the samples at 535 nm and detecting the emitted fluorescence at 590 nm (red filters) in a microwell plate in a fluorimeter capable of measuring fluorescence polarization.

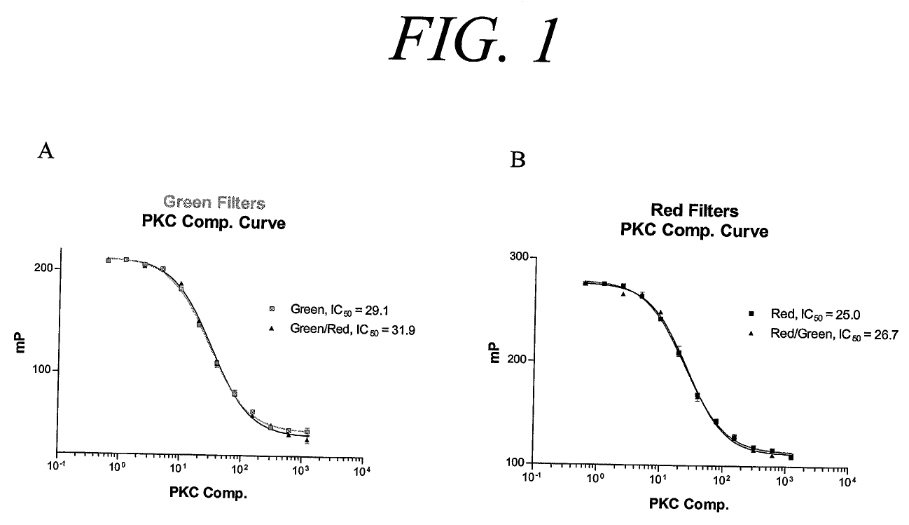

As shown in FIG. 1, the fluorescence polarization at high concentrations of phosphopeptide competitor was about 40 millipolarization units (mP) when measured using the green filters and about 100 mP when measured using the red filters. When the concentration of competitor was low, the mP increased significantly to ˜200 for the green filter measurement and to ˜270 in the red filter measurement, reflecting the fact that more of each of the labeled phosphopeptides bound to the antibody. These polarization measurements were not significantly different from the numbers obtained when performing the assay with only one color of fluorescently labeled phosphopeptide (1 nM) present per sample.

This assay may similarly be used to identify and characterize potential inhibitors of protein kinase enzyme reactions. In such a FP kinase assay, a kinase and a phosphorylatable peptide are incubated in a suitable buffer and the reaction is started by the addition of ATP. This reaction can occur in the presence of the anti-phosphopeptide antibody and fluorescently labeled-phosphopeptides, or, alternatively, the antibody and labeled-phosphopeptides can be added following the reaction. Potential inhibitors are added at various concentrations to the kinase reaction mixture prior to the addition of the ATP. The product of the kinase reaction—a non-labeled phosphopeptide—can compete with the labeled-phosphopeptides for binding to the antibody. The resulting FP value with then be low. If the compounds inhibit the kinase reaction, there is less or no non-labeled phosphopeptide produced, and therefore the labeled-phosphopeptides remain bound to the antibody. The resulting FP value is then higher than in the absence of inhibitor. Fluorescence polarization measurements in the presence of the suspected inhibitor are compared to duplicate samples lacking that suspected inhibitor. Many inhibitors could be screened using this method in a high-throughput format. In such an assay, any interference with FP by the inhibitor is overcome by using the two fluorophores to measure FP. This is because the inhibitor could only interfere at a wavelength corresponding to the absorption or emission optima of one of the two fluorophores.

### EXAMPLE 2

### Detection of Phosphorylated Peptide by Dual Color Fluorescence Polarization in the Presence of Contaminating Fluorescent Molecules

In the same fashion as in Example 1, samples containing of varying concentrations of competitor were incubated (for 2 hours at room temperature) with 6 nM PKC antibody, 0.75 nM fluorescein-labeled phosphopeptide, and 0.75 nM TMR-labeled phosphopeptide in the presence of increasing concentrations of either unconjugated fluorescein or TMR. Control wells were also prepared without either unconjugated fluorescein or TMR present. These fluorescent compounds were added to simulate contamination from an assay component that the present invention is designed to overcome.

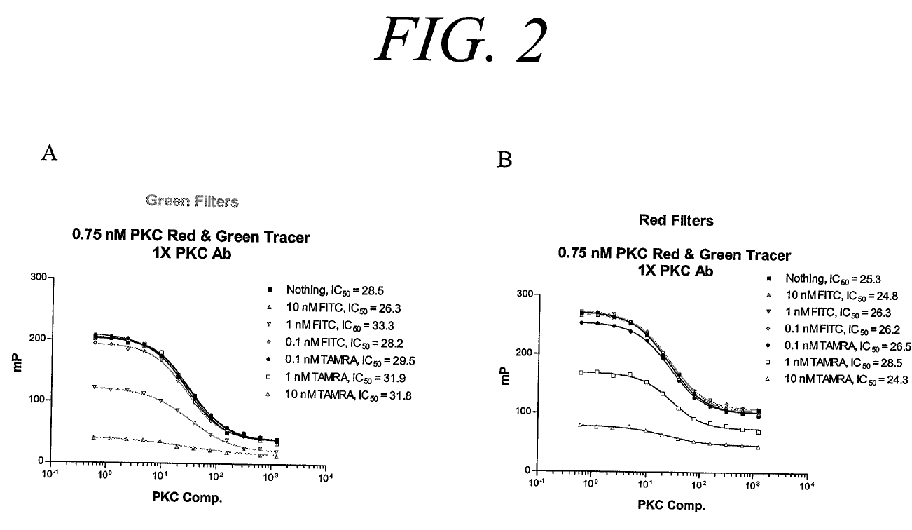

Two fluorescence polarization measurements were again obtained using the green and red filter pairs described in Example 1. As shown in FIG. 2, the dynamic range of the assay (change in mP from low competitor concentration to high competitor concentration) is decreased with increasing concentrations of the contaminating fluor corresponding to the filter set used to measure the polarization. That is, free fluorescein in the sample decreased the dynamic range when measuring the assay using the green filters, but not during the second measurement with the red filters. Both the fluorescence polarization and the fluorescence intensities were altered (as compared to the control wells) for the corresponding contaminating fluor-filter set used for measurement. This allows one to determine whether the first or second polarization measurement is closer to the control value.

Thus, the method is well suited to determine the amount of unlabeled competitor for binding to the binding partner present in the mixture (for example, an unlabeled phosphopeptide produced from a kinase reaction) in the presence of compounds that may themselves be fluorescent (for example, a percentage of compounds from a chemical library being screened as potential kinase inhibitors).

### EXAMPLE 3

### Nuclear Receptor Competitive Binding Assay by Dual Color Fluorescence Polarization Detection

A non-labeled steroid (dexamethasone) was serially diluted in 10 mM potassium phosphate pH 7.4,20 mM Na₂MoO₄, 2% DMSO, 0.1 mM EDTA, 0.5 mM DTT buffer containing 0.1 mM stabilizing peptide. This steroid was used as a competitive binder to glucocorticoid receptor (GR). The diluted dexamethasone was incubated for 2 hours at room temperature with 4 nM active GR, 0.8 nM Fluormone GS1 (a steroid labeled with fluorescein), and 0.8 nM Fluormone GS Red (a steroid labeled with a TMR derivative) in the presence of either unconjugated fluorescein or TMR. Control wells were also prepared without either unconjugated fluorescein or TMR present. As in Examples 1 and 2, two separate fluorescence polarization values were obtained for each sample using a green filter or a red filter.

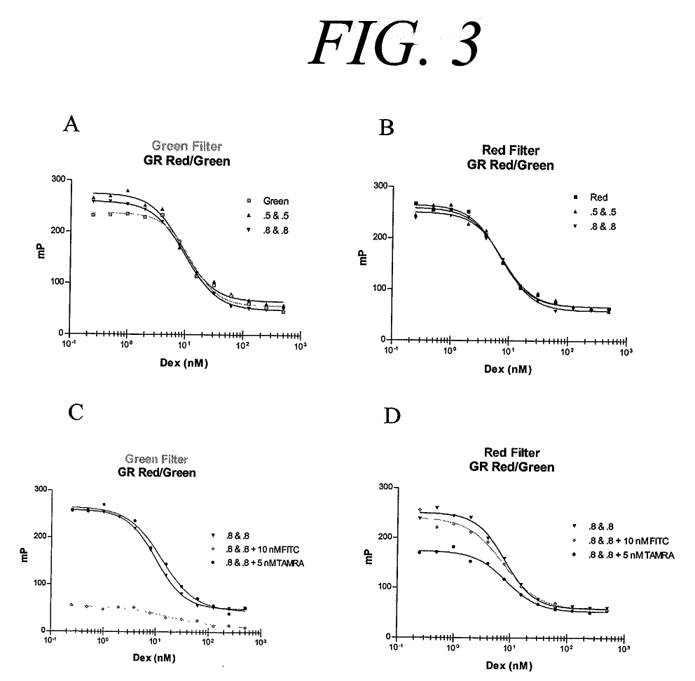

As shown, the fluorescence polarization at high concentrations of steroid competitor was about 60 millipolarization units (mP) (FIG. 3). When the concentration of competitor was low, the niP increased significantly (to ˜260 mP). As in Example 2, having free fluorescein present altered the fluorescence polarization measurements obtained using the green filter set, but did not affect the second fluorescence polarization measurements (red filter). The reverse was true of the samples containing free TMR. Thus, the method is well suited to the screening of chemical libraries for the presence of compounds that bind to the binding partner when such libraries contain compounds that are themselves fluorescent.

This assay may also be used to identify and characterize potential inhibitors of nuclear receptor-steroid binding. Such potential inhibitors are added at various concentrations to the mixture either prior to or following contact of the receptor with the labeled steroid. Fluorescence polarization measurements in the presence of the suspected inhibitor are compared to duplicate samples lacking that suspected compound. A screen is set up such that in the absence of the inhibitor, the Fluormone GS 1:Glucocorticoid Receptor and the Fluormone GS Red:Glucocorticoid Receptor complexes remain intact, yielding high polarization values. If the inhibitor can compete for binding, the fluorophore-labeled steroids are released from the glucocorticoid receptor and have a low polarization value. In such an assay, any interference with FP by the inhibitor is overcome by using the two fluorophores to measure FP. This is because the inhibitor could only interfere at a wavelength corresponding to the absorption or emission optima of one of the two fluorophores.

### EXAMPLE 4

### Detecting Nucleic Acid Cleavage using Fluorescence Polarization

The following method is described in more detail in U.S. Pat. No. 5,786,139, which is herein incorporated by reference. This experiment is performed as described in the patent except that the nucleic acid is labeled with two fluorophores. The two fluorophores, fluorescein and tetramethylrhodamine, are attached to the nucleic acid so that resonance energy transfer is less than 50% in the construct. Labeling the nucleic acid with two specific fluorophores can be done in several ways, including 1) doing chemical nucleic acid syntheses in which the fluorophores are incorporated at specific positions, or 2) enzymatic synthesis where the concentration of the labeled nucleotides versus unlabeled nucleotide are controlled so that the labeled nucleotides are incorporated far enough apart in so that there is minimal or no FRET. The fluorescence polarization assay is then performed as described in the patent, except that the polarization results are measured independently at the two wavelengths corresponding to the emission wavelengths of each fluorophore.

Alternatively, a subpopulation of nucleic acid is labeled with fluorescein and another subpopulation is labeled with tetramethylrhodamine. The subpopulations are combined and then the experiment is carried out as described above.

More specifically, the following assay is used to detect RNase activity. Both end-point and kinetic RNase activity assays are performed using a modification of the Beacon® RNase Activity Detection Kit (PanVera® Part No. P2115). In the end-point assay, RNase A (Sigma Chemical Co., St. Louis, Mo.) is diluted into a 100 μL volume of 25 mM Tris-HCl (pH 7.8), 5 mM MgCl₂ in RNase free (DEPC-treated) water. The assays are started by addition of 25 ng fluorescein-labeled RNA (Fl-RNA; 900 bases) and 25 ng of tetramethylrhodamine-labeled RNA (TMR-RNA; 900 bases). Reactions are incubated at 37° C. for 1 hour in a water bath, and are quenched by adding a 50 μL aliquot of each reaction mixture to a tube containing 1.1 mL of 78 mM Tris-HCl (pH 8.0), 0.124% SDS. The fluorescence polarization value in each reaction is measured on the Beacon® Analyzer using appropriate excitation and emission filters for the respective fluorophores and compared to a sample containing all of the above components except RNase. The reduced FP values in the RNase-containing samples indicate that labeled RNA sample is being digested. Alternatively, the assay can be carried out using a single sample of RNA labeled with both fluorescein and tetramethylrhodamine.

Kinetic RNase assays are performed by adding 25 ng Fl-RNA and 25 ng of TMR-RNA to 1.1 mL of 25 mM Tris-HCl (pH 7.8), 5 mM MgCl₂. Various amounts of RNase A (0.5-20 ng) are added to the reaction tube and the fluorescence polarization was measured every 13 seconds for 20 minutes on a Beacon® Analyzer. Alternatively, a sample of RNA labeled with both fluorophores could be substituted. As above, changes in fluorescence polarization between each nuclease sample and a reagent blank (all components except the RNase) were used to measure enzyme activity.

End-point DNase activity assays are performed using a modification of the Beacon® DNase Activity Detection Kit (PanVera® Part No. P2012). DNase I is obtained from Sigma Chemical Company (St. Louis, Mo.), and Exonuclease III from New England BioLabs (Beverly, Mass.). Various concentrations of the two enzymes are diluted into a 40 μL volume of 25 mM Tris-HCl (pH 7.8), 5 mM MgCl₂, 0.05% Triton® X-100 in DNase free water. The assays are started by addition of 3 ng fluorescein-labeled DNA and 3 ng of tetramethylrhodamine-labeled DNA in 10 μL DNase Assay Buffer. Reactions are incubated at 37° C. for 1 hour in a water bath, and reactions are quenched by adding each 50 μL reaction mixture to a tube containing 1 mL of 100 mM Tris-HCl (pH 8.0), 5 mM MgCl₂, 0.5% SDS. The fluorescence polarization value in each reaction is measured on the Beacon® Analyzer using appropriate excitation and emission filters for the respective fluorophores and compared to a sample containing all of the above components except DNase and Exonuclease III. The reduced FP values in the DNase-containing samples indicate that labeled DNA sample is being digested. Alternatively, the assay can be carried out using a single sample of DNA labeled with both fluorescein and tetramethylrhodamine.

Both the RNase and the DNase/Exonuclease III assays may also be used to identify and characterize potential enzyme modulators. Such potential modulators are added at various concentrations to the enzyme either prior to or following contact with the labeled casein substrate. Fluorescence polarization measurements in the presence of the suspected modulator are compared to duplicate samples lacking that suspected modulator. In such an assay, any interference with FP by the modulator is overcome by using the two fluorophores to measure FP. This is because the modulator could only interfere at a wavelength corresponding to the absorption or emission optima of one of the two fluorophores. If the suspected modulator is an inhibitor of the enzyme, the FP of the sample containing that inhibitor will be higher than the sample containing enzyme, but no suspected modulator. If the suspected modulator is an enhancer, the FP of the sample containing that enhancer will be lower than the sample containing enzyme, but no suspected modulator.

### EXAMPLE 5

### Detecting Protease Activity using Fluorescence Polarization

The general method for monitoring proteolytic cleavage by fluorescence polarization is described in U.S. Pat. No. 5,804,395, which is herein incorporated by reference. This assay may be carried out in either of two ways according to the present invention. In one assay, a subpopulation of substrate is labeled with fluorescein and another subpopulation of substrate is labeled with tetramethyrhodamine. After purification of the fluorophore-labeled substrates, the two subpopulations are mixed and used in protease assay to be monitored by fluorescence polarization. In the second assay, the substrate is labeled with two different fluorophores incorporated onto the same substrate molecule. The two labeling reactions are performed in the same tube simultaneously or consecutively.

Specific fluorescence polarization-based protease assays are performed using a modification of the Beacon® Protease Activity Detection Kit (PanVera®, Madison, Wis., Part No. P2010). All enzymes (Sigma Chemical Co., St. Louis, Mo.) used in this assay are diluted into a 1 mL volume in 12×75 mm disposable borosilicate test-tubes (PanVera® Part No. P2182) at their optimum pH: kallikrein, elastase in 10 mM Tris-HCl (pH 8.8); trypsin, thrombin, plasmin, chymotrypsin, thermolysin, and pronase in 10 mM Tris-HCl (pH 7.8); papain in 10 mM Tris-HCl (pH 6.2); cathepsin B in 10 mM sodium acetate, 4 mM EDTA, 5 mM cysteine (pH 5.2); pepsin in 10 mM HCl (pH 2.0). The assays are started by the addition of 5 pmoles of fluorescein-labeled casein (FTC-casein) and 5 pmoles of tetramethylrhodamine-labeled casein (TMR-casein). The tubes are then incubated at 37° C. in a water bath. After 1 hour, the reactions are stopped by the addition of 200 μL 0.5 M Tris-HCl (pH 8.8). The outside surfaces of the test tube are wiped dry. Changes in fluorescence polarization between each protease sample and a blank containing all reagents except the protease are read on a Beacon® 2000 Fluorescence Polarization Instrument (PanVera® Part No. P2300) utilizing excitation and emission filters for each of the respective fluorophores. A reduction in the FP of samples containing enzyme indicates that the enzyme has digested the labeled casein substrate. The identical assay can also be performed using casein labeled with both tetramethylrhodamine and fluorescein.

To identify and characterize potential modulators of any of the above enzyme, compounds suspected of being a modulator are added at various concentrations to the enzyme either prior to or following contact with the labeled casein substrate. Fluorescence polarization measurements in the presence of the suspected modulator are compared to duplicate samples lacking that suspected modulator. In such an assay, any interference with FP by the modulator is overcome by using the two fluorophores to measure FP. This is because the modulator could only interfere at a wavelength corresponding to the absorption or emission optima of one of the two fluorophores. If the suspected modulator is an inhibitor of the enzyme, the FP of the sample containing that inhibitor will be higher than the sample containing enzyme, but no suspected modulator. If the suspected modulator is an enhancer, the FP of the sample containing that enhancer will be lower than the sample containing enzyme, but no suspected modulator.

Alternatively, the source of the enzyme may be a biological sample, rather than an isolated preparation. That biological sample may also contain molecules that interfere with FP. Again the use of two fluorophores overcomes any interference.

### EXAMPLE 6

### Protein Tyrosine Phosphatase Assays

The enzymatic activity of T-Cell Protein Tyrosine Phosphatase (TC PTP) was measured in a dose-dependent manner by incubating different concentrations of TC PTP (New England Biolabs; Beverly, Mass.), from 0.05 U/μL to 0.0005 U/μL, with 50 μL of a mixture of an anti-phosphotyrosine antibody, a fluorescein-labeled phosphopeptide and a tetramethylrhodamine-labeled phosphopeptides. The final reaction volume was 1100 L. Two control assays were performed. One did not receive TC PTP, while the other was supplemented with 50 μM Na₃VO₄ (Sigma, St. Louis, Mo.), a potent phosphatase inhibitor. Fluorescence polarization measurements were performed using the appropriate excitation and emission filters appropriate for each fluorophore. Increasing concentrations of TC PTP result in an increased rate of dephosphorylation of the labeled phosphopeptides, which is indicated by a decrease in polarization (due to the inability of the dephosphorylated peptide to bind to the antibody). This change in polarization was dependent on the presence of TC PTP and could be completely inhibited by 50 μM Na₃VO₄.

To demonstrate the usefulness of an FP-based phosphatase assay in screening for phosphatase inhibitors, the IC₅₀ value for Na₃VO₄ and TC PTP was determined. In a black, 96-well plate (DYNEX; Chantilly, Va.), Na₃VO₄ (Sigma; St. Louis, Mo.) was serially diluted 2-fold into 24 wells from a starting concentration of 500 nM in a volume of 50 μL. TC PTP was then added to each well so that the final concentration of the enzyme would be 0.005 U/μL per assay. To start the reaction, 50 μL of a mixture of anti-phosphotyrosine antibodies and fluorescein-labeled phosphopeptides and tetramethylrhodamine-labeled phosphopeptides was added, and each reaction was incubated for 15 minutes. At the end of the incubation, 10 μL of 550 μM Na₃VO₄ was added to quench all of the reactions, and the polarization for each sample was measured on a TECAN Polarion using the appropriate excitation and emission filters appropriate for each fluorophore. Nonlinear regression analysis of a semi-log plot was used to analyze these data. The IC₅₀ of the ubiquitous phosphatase inhibitor Na₃VO₄ was determined to be 4 nM for TC PTP.

In each of the above experiments, phosphopeptides could be alternatively labeled with two fluorophores (such as fluorescein and cy7) positioned on the phosphopeptide substrate so that fluorescence energy transfer was minimized.

While we have herein before presented a number of embodiments of this invention, it is apparent that our basic construction may be altered to provide other embodiments that utilize the methods and kits of this invention. Therefore, it will be appreciated that the scope of this invention is to be defined by the appended claims, rather than by the specific embodiments that have been presented by way of example.

We claim:

1. A method of determining by fluorescence polarization if a population of compounds binds to a binding partner, said method comprising the steps of:
a. providing a mixture comprising at least two subpopulations of compounds, wherein:
i. each subpopulation is characterized by a single compound labeled with a fluorophore;
ii. the fluorophore in any subpopulation has a maximal excitation peak that is at least 30 nanometers different from the maximal excitation peak of the fluorophore in another subpopulation and a maximal emission peak that is at least 30 nanometers different from the maximal emission peak of the fluorophore in another subpopulation; and
iii. the labeled compound in each of the subpopulations has a dissociation constant for binding to said binding partner that is less than 50-fold different from the dissociation constant for the binding of the labeled compound in any other of the subpopulations; and
b. comparing the fluorescent polarization values of said mixture in the absence and the presence of said binding partner at multiple wavelengths that correspond to the wavelengths at which each of said fluorophores present in said mixture optimally absorbs and emits.

2. A method of determining by fluorescence polarization if a compound binds to a binding partner, said method comprising the steps of:
a. providing a solution comprising said compound labeled with at least two different fluorophores, wherein:
i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound, and
ii. there is less than 50% fluorescence resonance energy transfer between any two of the fluorophores present in the labeled compound; and
b. comparing the fluorescent polarization values of said solution in the absence and the presence of said binding partner at multiple wavelengths that correspond to the wavelengths at which each of said fluorophores present on said compound optimally absorbs and emits.

3. A method of determining by fluorescence polarization if a population of compounds is incorporated into a larger molecule, said method comprising the steps of:
a. providing a first mixture comprising at least two subpopulations of compounds, wherein:
i. each subpopulation is characterized by a single compound labeled with a fluorophore;
ii. the fluorophore in any subpopulation has a maximal excitation peak that is at least 30 nanometers different from the maximal excitation peak of the fluorophore in another subpopulation and a maximal emission peak that is at least 30 nanometers different from the maximal emission peak of the fluorophore in another subpopulation; and
iii. the labeled compound in each of the subpopulations is capable of being incorporated into a larger molecule through the action of an enzyme or a catalyst; and
iv. the labeled compound in each of the subpopulations has a rate of incorporation into a larger molecule through the action of said enzyme or said catalyst that is less than 50-fold different from the rate of incorporation into a larger molecule by said enzyme or said catalyst of the labeled compound in any other of the subpopulations;
b. measuring the fluorescence polarization values of said first mixture at multiple wavelengths that correspond to the wavelengths at which each of said fluorophores present in said first mixture optimally absorbs and emits;
c. adding to said first mixture a solution comprising said enzyme or said catalyst capable of incorporating said compounds into a larger molecule to create a second mixture, wherein said second mixture is incubated for a period of time and provides conditions that, in the absence of an inhibitor of said enzyme or said catalyst, allow said compounds to be incorporated into a larger molecule through the action of said enzyme or said catalyst;
d. measuring the fluorescence polarization values of said second mixture at multiple wavelengths that correspond to the wavelengths at which each of said fluorophores present in said mixture optimally absorbs and emits;
e. comparing the fluorescence polarization measurements taken at steps b and d to determine if said population of compounds have been incorporated into a larger molecule.

4. A method of determining by fluorescence polarization if a compound is incorporated into a larger molecule, said method comprising the steps of:
a. providing a first solution comprising a compound labeled with at least two different fluorophores, wherein:
i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound;
ii. there is less than 50% fluorescence resonance energy transfer between any two of the fluorophores present in the labeled compound; and
iii. said compound is capable of being incorporated into a larger molecule through the action of an enzyme or a catalyst;
b. measuring the fluorescence polarization values of said first solution at multiple wavelengths that correspond to the wavelengths at which each of said fluorophores present on said compound optimally absorbs and emits;
c. adding to said first solution said a solution comprising said enzyme or said catalyst capable of incorporating said compounds into a larger molecule to create a second solution, wherein said second mixture is incubated for a period of time and provides conditions that, in the absence of an inhibitor of said enzyme or said catalyst, allow said compound to be incorporated into a larger molecule through the action of said enzyme or said catalyst;
d. measuring the fluorescence polarization values of said second solution at multiple wavelengths that correspond to the wavelengths at which each of said fluorophores present in said mixture optimally absorbs and emits; and
e. comparing the fluorescence polarization measurements taken at steps b and d to determine if said compound has been incorporated into a larger molecule.

5. A method of determining by fluorescence polarization if a population of compounds is degraded into smaller molecules, said method comprising the steps of:
a. providing a first mixture comprising at least two subpopulations of compounds, wherein:
i. each subpopulation is characterized by a single compound labeled with a fluorophore;
ii. the fluorophore in any subpopulation has a maximal excitation peak that is at least 30 nanometers different from the maximal excitation peak of the fluorophore in another subpopulation and a maximal emission peak that is at least 30 nanometers different from the maximal emission peak of the fluorophore in another subpopulation;
iii. the labeled compound in each of the subpopulations is capable of being degraded into smaller molecules through the action of an enzyme; and
iv. the labeled compound in each of the subpopulations has a rate of degradation through the action of said enzyme that is less than 50-fold different from the rate of degradation by said enzyme of the labeled compound in any other of the subpopulations;
b. measuring the fluorescence polarization values of said first mixture at multiple wavelengths that correspond to the wavelengths at which each of said fluorophores present in said first mixture optimally absorbs and emits;
c. adding to said first mixture a solution comprising said enzyme capable of degrading said compounds into smaller molecules to create a second mixture, wherein said second mixture is incubated for a period of time and provides conditions that, in the absence of an inhibitor of said enzyme, allow said compounds to be degraded into smaller molecules through the action of said enzyme;
d. measuring the fluorescence polarization values of said second mixture at multiple wavelengths that correspond to the wavelengths at which each of said fluorophores present in said mixture optimally absorbs and emits; and
e. comparing the fluorescence polarization measurements taken at steps b and d to determine if said population of compounds have been degraded into smaller molecules.

6. A method of determining by fluorescence polarization if a compound is degraded into smaller molecules, said method comprising the steps of:
a. providing a first solution comprising a compound labeled with at least two different fluorophores, wherein:
i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound;
ii. there is less than 50% fluorescence resonance energy transfer between any two of the fluorophores present in the labeled compound; and
iii. said compound is capable of being degraded into smaller molecules through the action of an enzyme;
b. measuring the fluorescence polarization values of said first solution at multiple wavelengths that correspond to the wavelengths at which each of said fluorophores present on said compound optimally absorbs and emits;
c. adding to said first solution a solution comprising said enzyme capable of degrading said compound into smaller molecules to create a second solution, wherein said second mixture is incubated for a period of time and provides conditions that, in the absence of an inhibitor of said enzyme, allow said compound to be degraded into smaller molecules through the action of said enzyme;
d. measuring the fluorescence polarization values of said second solution at multiple wavelengths that correspond to the wavelengths at which each of said fluorophores present in said mixture optimally absorbs and emits; and
e. comparing the fluorescence polarization measurements taken at steps b and d to determine if said compound has been degraded into smaller molecules.

7. The method according to claim 1 or 2, wherein said fluorescence polarization values in the presence of said binding partner are determined in the presence of a substance suspected of being a modulator of the ability of said binding partner to bind to said labeled compounds or compound.

8. The method according to claim 1 or 2, wherein said fluorescence polarization values in the presence of said binding partner are determined in the presence of a molecule that inhibits the ability of said binding partner to bind to said labeled compound or compounds and a substance suspected of being a modulator of said molecule, and after said molecule and said substance have been contacted with one another for a time and under conditions that would allow a modulator to affect said molecule.

9. The method according to claim 1 or 2, wherein said fluorescence polarization values in the presence of said binding partner are determined in the presence of an enzyme, a substrate for said enzyme and a substance suspected of being a modulator of said enzyme, and after said enzyme, said substrate and said suspected inhibitor have been incubated together for a time and under conditions that, in the absence of a modulator of said enzyme, allow said enzyme to act upon said substrate to produce a product that inhibits the ability of said binding partner to bind to said labeled compound or compounds.

10. The method according to claim 3 or 4, wherein prior to step d, a substance suspected of modulating the ability of said enzyme or catalyst to incorporate said labeled compound or compounds into a larger molecule is contacted with said labeled compound, said enzyme or said catalyst for a time and under conditions that would allow said substance to interact with said labeled compound or compounds, said enzyme or said catalyst and modulate the ability f said enzyme or catalyst to incorporate said labeled compound or compounds into a larger molecule.

11. The method according to claim 5 or 6, wherein prior to step d, a substance suspected of modulating the ability of said enzyme to degrade said labeled compound or compounds is contacted with said labeled compound or compounds or said enzyme for a time and under conditions that would allow said substance to interact with said labeled compound or compounds or said enzyme and modulate the ability of said enzyme to degrade said labeled compound or compounds.

12. The method according to any one of claims 2, 4 or 6, wherein each of said fluorophores is conjugated to one another.

13. The method according to any one of claims 2, 4 or 6, wherein there is less than 30% fluorescence resonance energy transfer between any two of the fluorophores present in the labeled compound.

14. The method according to claim 13, wherein there is less than 5% fluorescence resonance energy transfer between any two of the fluorophores present in the labeled compound.

15. The method according to any one of claims 1 to 6, wherein one of said fluorophores is a green fluorophore having an emission maxima between 480 and 530 nm and another of said fluorophores is a red fluorophore having an emission maximum of greater than 570 nm.

16. The method according to claim 15, wherein said green fluorophore is fluorescein and said red fluorophore is a tetramethylrhodamine dye or a cyanine dye.

17. The method according to claim 1 or 2, wherein each of said compounds is a phosphopeptide, and said binding partner binds to said compound through a phosphorylated amino acid present in said phosphopeptide.

18. The method according to claim 17, wherein said binding partner is a protein which binds to said phosphopeptide, but does not bind to a dephosphorylated form of said phosphopeptide.

19. The method according to claim 1 or 2, wherein each of said compounds is a steroid or a steroid mimic and said binding partner is a nuclear receptor.

20. A kit comprising:
a. a first subpopulation of a compound labeled with a first fluorophore;
d. a second subpopulation of a compound labeled with a second fluorophore that has a maximal excitation peak that is at least 30 nanometers different from the maximal excitation peak of the fluorophore in said first subpopulation and a maximal emission peak that is at least 30 nanometers different from the maximal emission peak of the fluorophore in said first subpopulation, wherein said compound in said second subpopulation has:
i. a dissociation constant for binding to a binding partner that is less than 50-fold different from the dissociation constant for the binding to said binding partner by said compound in said first subpopulation, or
ii. a rate of incorporation into a larger molecule by an enzyme or a catalyst that is less than 50-fold different from the rate of incorporation into a larger molecule by said enzyme or said catalyst of said compound in said first subpopulation, or
i. a rate of degradation by an enzyme that is less than 50-fold different from the rate of degradation by said enzyme of said compound in said first subpopulation; and
b. instructions for using said kit which instruct the user to perform fluorescence polarization measurements on a mixture comprising said first and said second subpopulations of compound at multiple wavelengths corresponding to the wavelengths at which each of said first and second fluorophores emits.

21. The kit according to claim 20, wherein the compound labeled with said first fluorophore and the compound labeled with said second fluorophore are pre-mixed in a single vessel.

22. The kit according to claim 20, additionally comprising:
d. an enzyme or catalyst capable of incorporating said compound in said first subpopulation and said compound in said second subpopulation into a larger molecule, wherein said compound in said second subpopulation has a rate of incorporation into a larger molecule by said enzyme or said catalyst that is less than 50-fold different from the rate of incorporation into a larger molecule of said compound in said first subpopulation by said enzyme or said catalyst, or
e. an enzyme capable of degrading said compound in said first subpopulation and said compound in said second subpopulation into smaller molecules, wherein said compound in said second subpopulation has a rate of degradation by said enzyme that is less than 50-fold different from the rate of degradation by said enzyme of said compound in said first subpopulation, or
a. a binding partner, wherein said compound in said second subpopulation has a dissociation constant for binding to said binding partner that is less than 50-fold different from the dissociation constant for the binding to said binding partner by said compound in said first subpopulation.

23. A kit comprising:
a. a compound labeled with at least two fluorophores, wherein:
i. each fluorophore has a maximal excitation peak that is at least 30 nanometers different from the maximal excitation peak of another fluorophore on said compound and a maximal emission peak that is at least 30 nanometers different from the maximal emission peak of another fluorophore on said compound, and
ii. there is less than 50% fluorescence resonance energy transfer between any two of the fluorophores present in the labeled compound; and
b. instructions for using said kit which instruct the user to perform fluorescence polarization measurements on a solution comprising said compound at multiple wavelengths corresponding to the wavelengths at which each of said fluorophores emits.

24. A kit comprising:
a. a compound;
b. a first fluorophore;
c. a second fluorophore that has a maximal emission peak that is at least 30 nanometers different from the maximal absorbance peak of the first fluorophore, wherein there is less than 50% energy transfer between said first and said second fluorophores when said compound is labeled with said fluorophores; and
d. instructions for using said kit which instruct the user to:
i. label said compound with said first and said second fluorophore;
ii. perform fluorescence polarization measurements on a solution comprising said labeled compound at multiple wavelengths corresponding to the wavelengths at which each of said fluorophores emits.

25. The kit according to claim 23 or 24, additionally comprising:
a. a binding partner capable of binding said compound, or
b. an enzyme capable of incorporating said compound into a larger molecule, or
c. or an enzyme capable of degrading said compound into smaller molecules.

26. The kit according to claim 24, wherein said compound, said first fluorophore and said second fluorophore are each in separate vessels.

27. The kit according to claim 23 or 24, wherein said first fluorophore is conjugated to said second fluorophore.

28. The kit according to claim 22, wherein said kit additionally comprises a binding partner and the compound and the binding partner are pre-mixed in a single vessel.

29. The kit according to claim 25, wherein said kit additionally comprises a binding partner and the compound and the binding partner are pre-mixed in a single vessel.

30. The kit according to any one of claims 20, 23 or 24, wherein one fluorophore is fluorescein and another fluorophore is a tetramethylrhodamine dye or a cyanine dye.

31. The kit according to claim 23 or 24, wherein there is less than 30% energy transfer between said first and said second fluorophores when said compound is labeled with said fluorophores.

32. The kit according to claim 31, wherein there is less than 5% energy transfer between said first and said second fluorophores when said compound is labeled with said fluorophores.
